(19) United States
(12) Patent Application Publication
(10) Pub. No.: US 2024/0082554 A1
(43) Pub. Date: Mar. 14, 2024

(54) PELLET INSERTION DEVICE

(71) Applicant: Intellipelle, LLC, LIbertyville, IL (US)

(72) Inventors: Mishail A. Shapiro, LIbertyville, IL (US); Alex Oksengendler, Mundelein, IL (US)

(21) Appl. No.: 17/712,136

(22) Filed: Apr. 2, 2022

(51) Int. Cl. A61M 37/00

(52) U.S. Cl. CPC A61M 37/0069; A61M 2209/06

(57) ABSTRACT

An atraumatic insertion device for the subcutaneous delivery of pellet-shaped medicaments and/or implantable devices is described. The device includes a cannula with a distal tip that opens when the pellet-shaped medicaments and/or implantable devices are pushed through using an obturator. The cannula distal tip has at least three cuts, creating sectional leaves that open when pressure is applied distally from within the hollow cannula body. The distal tip of the obturator has complementary grooves to the sectional leaves of the cannula distal tip and will force everything out of the cannula and anything that might be caught on the sectional leaves. A blister packaging strip for storing and dispensing pellet-shaped medicaments and/or implantable devices is used to load the insertion device. The blister packaging strip has features to align the blister cavity directly above the loading well of the device and safely transfer the pellet-shaped medicaments and/or implantable devices into the cannula.

### FIELD

This invention is directed to a device that inserts medicated pellets and/or implantable devices subcutaneously into humans or animals. More particularly, the invention relates to improvements in the components of the insertion device, and the packaging used to load the pellets/implants into the insertion device.

### BACKGROUND

There exists a need for an improved device to insert subcutaneous medicaments and/or implantable devices. Synthetically designed subcutaneous pellets are used as medicaments by medical professionals for a variety of issues. For example, both biosimilar and bioidentical hormone replacement therapy utilizes subcutaneous pellet placement as the preferred delivery method when compared to oral and injectable administration. Opioid, antibiotic, diabetes, and cardiac treatments may soon become available in a subcutaneous tissue delivery method as well.

Subcutaneous pellet medicament formulations are created either by pressure treatments/sterilization protocol or in combination with an organic matrix to allow for controlled release of said medicaments directly into the bloodstream. Subcutaneous pellets can be formulated for a slow-release administration that takes place over a period of weeks or months as opposed to hours for oral medicaments.

Subcutaneous pellets have practical advantages for human and animal patients over patches, creams, and injections. The controlled-release formulation circumvents the “first-pass” metabolism of the hepatic breakdown, does not affect clotting factors, and does not increase the risk of thrombosis. The release of the drug from implanted pellets may continue for a period of three to six months depending on the size and composition of the pellet. This greatly reduces trips to the doctor for injections, and eliminates adherence concerns typical with patient-administered medicaments. Additionally, pellet therapy keeps hormone levels consistent throughout the day and avoids rollercoaster-like effects from oral, topical, and injection administrations.

Subcutaneously implanted hormone pellets may be smaller than a grain of rice and are implanted into the subcutaneous tissue where they provide a slow continuous release of hormone(s) into the bloodstream. Typically, the pellets are implanted in the lower abdomen or buttocks. The procedure is done in a physician's office with the use of a local anesthetic and a small incision for insertion of a cannula.

Civilian populations without access to adequate nearby healthcare can greatly benefit from this medication delivery method. Military populations can also benefit when medical staff attend to a large number of individuals in need of immediate treatment but staffers are too busy to promptly switch intravenous bags containing antibiotics or other life sustaining interventions. Additionally, one can imagine many spheres of activity such as adventuring in remote locations wherein prolonged medication delivery in less-than-ideal circumstances is required and professional medical care is not readily accessible.

Another use for such a device is to insert subcutaneous devices/implants into humans or animals. Electronic “personal” identification tracking devices and identification “badges” are approximately the same size and shape as medicinal pellets, and can be safely stored in a subcutaneous location. Innovative and cutting-edge technologies such as Radio Frequency Identification (“RFID”) have been successfully integrated into healthcare environments to reduce adverse events by quickly accessing the unambiguous identification of the patient, medical history, current medication (name, lot number, expiry date, indications for administration and storage, etc.) and tracking throughout a healthcare setting during an extended visit. Additionally, data analysis of the RFID register can measure the efficiency of the medical services and detect bottlenecks so that actions can be undertaken to improve weak points in the process.

Other types of subcutaneous devices have been envisioned in the healthcare setting. For example, implantable miniature physiologic monitoring devices that allow for blood collection and analysis with the ability to communicate electronically. Wearable devices for blood sugar monitoring already exist, and it is only a matter of time before they are miniaturized and implantable. Any device capable of biometric testing and reduced to the size of a grain of rice would be beneficial in both civilian and military populations.

Moreover, animals have been implanted with RFID microchips since the 1980s to help identify their names, owners, and medical history. The microchip is encapsulated in biocompatible glass (the type used in pacemakers, stents and other medical devices), and has been tested to last for at least 75 years.

In practice, the subcutaneous implant device is preferably used in a professional medical setting. Based on the patient's occupation (i.e., standing versus sitting occupation), the area of subcutaneous insertion is identified either above or below the patient's belt. A sterile marker identifies the point of insertion medially and a five-centimeter line is drawn laterally. Preferably, a local anesthetic is administered in a transcutaneous plane along the markings prior to the incision. A lancet attached to the insert device invention is used to create an incision along the mark to a depth of approximately two centimeters utilizing a depth gauge that prevents penetration deeper than intended. The exposed lancet is covered to prevent unintentional incisions and used as a tissue placement guide during the insertion procedure.

The blister packaging strip of pellets is placed into the locking clips of the invention, and the desired number of sterile pellets are ejected from the sterile packaging directly into the loading well. The locking clips can be as simple as an L-shaped arm with a gap slightly larger than the width of the flat edge of the blister packaging strip. Either gravity, the obturator, or both are used to advance the pellets into the hollow cannula body. Once the desired number of pellets is loaded into the hollow passageway of the cannula, a thumb-grip slide advances the cannula-holder forward to compress a spring inside the outer body to engage with a releasable-locking mechanism. The cannula-holder slides along the longitudinal axis of the cannula inside the outer body on a track or groove. The loading well, which is attached to the cannula-holder thumb-grip, seals shut as it slides under the top cover of the device. The locking clip attached to the thumb-grip will release the blister packaging strip of pellets as it is advanced forward.

Now that the cannula is fully extended and securely locked in its most forward position, the obturator must also be fully advanced and locked into place using a releasable-locking mechanism in the back cover of the device coupled with the proximal end of the obturator near its grip.

The user can now begin the insertion procedure. One hand of the user grasps the patient's skin with the thumb and forefinger to slightly pinch the incision so that the cannula distal tip can be inserted. The depth gauge covering the lancet prevents the cannula from being inserted more than approximately two centimeters into the subcutaneous tissue by using the skin as a roadblock. Due to the position of the depth gauge on the top cover of the device, the user can use it as a lever to angle the bore, thus redirecting the cannula subcutaneously to assure proper placement of one or more pellets. The patient's skin would then stop the cannula from being inserted deeper as it comes in contact between the depth gauge and the front cover of the device.

The blunt, atraumatic tip of the cannula is designed to push the subcutaneous vascular structures away rather than slicing through them. The trans-planar transgression of the angled bore hole is less likely to cause trauma compared to a sharpened cannula tip, thus minimizing possible organ injury during the procedure. Most importantly, the atraumatic tip, which curves inward in the direction of the longitudinal axis of said cannula, has at least three straight-cut sections along the longitudinal axis, resulting in a corresponding number of distal inward-curving tip sections, or “leaves.”

Once the cannula is fully inserted into the proper boring position, the user pushes the release button for the compressed spring that locked the cannula in a forward position. The cannula retracts to its original position, leaving the obturator locked in place. Notably, the length of the locked obturator is longer than the retracted cannula. The pellets are held firmly in place by the locked obturator as the cannula distal tip retracts. This causes the flexible sectional leaf tips of the cannula to open due to the internal pressure of the pellets and obturator pushing against the coiled spring elongation. The result is one or more perfectly placed pellets aligned in the bore hole. Importantly, the opening and closing of the cannula tip sectional leaves do not cause any damage to the pellets or implantable devices.

The obturator's distal tip is designed with grooves to fit between the sectional leaves of the cannula distal tip as it is retracted. Any subcutaneous tissue that entered the cannula during the insertion procedure is removed by the obturator and its grooved distal tip as the cannula retracts to its original position. After the cannula is returned to its original position, the obturator will also be automatically unlocked. Alternately, the obturator can be manually unlocked and retracted using its grip, or the entire device can be slowly lifted from the patient allowing the obturator to be withdrawn from the boring hole. Once the grooved distal tip of the obturator is removed from the distal tip opening of the cannula, the sectional leaves of the cannula tip will return to their original position.

When appropriate, the device can be reused utilizing the original incision for a second dosage of one or more pellets. The second dosage insertion point should be angled at least fifteen to thirty degrees away from the first insertion point boring line, and the entire process should be repeated.

The patient's incision should be addressed once the insertion procedure is completed. The incision needs to be sealed sanitarily using appropriate medical procedures. For example, Steri-Strips can be placed on the incision and covered by a gauze pad with an occlusive dressing.

To prevent the reuse of a discarded device, one or more safety features are implemented. For example, the obturator can be designed to easily break and prevent further use.

Under certain circumstances, the entire pellet insertion procedure can be self-administered using this device. The pellets are alternately deposited in a subcutaneous position in the abdominal fat or a lateral flank.

Unlike the present device with a blunt, atraumatic cannula tip, a patient's primary response to the traumatic cutting insertion of the prior art beveled trocar results in inflamed tissue, lymph fluid, and clotted red blood cells. Prior art traumatic trocar insertion is painful and results in scarring. Traumatically inserted pellets may lead to infection and often pass outside the body, which requires replacement with an additional traumatic insertion. The patient's inflammatory response to the traumatic insertion causes patients significant pain in the days following insertion. Moreover, the cutting and spearing motions used to insert beveled trocars cause significant bruising immediately after insertion that lasts for days or weeks, and cause scarring that may remain for a year or more. This inflammatory response increases the healing time of the incision, and increases the probability that one or more pellets may extrude due to external pressures (e.g., falling on, sitting on, or bumping the insertion region) or internal pressures (e.g., strenuous exercise or muscle contraction).

Review of prior art reveals noticeable differences from the present invention. The vast majority use a “push-through” method in which the obturator is used to manually deposit the pellets by pushing them through the cannula or trocar. This method can crush biologically active pellets, causing an unintended increase in the absorption rate of the medicament and result in patient overdose. It can also cause misalignment of the pellets, which predisposes pellet extrusion and a need to repeat the procedure. Most prior art also require pellets to be individually loaded into the needle or cannula by hand, increasing the chance for contamination of a sterile procedure and limiting the usefulness of the device to veterinary applications only. Additionally, the distal tips of prior art needles and cannulas are wide open and allow pellets to egress using only gravity. Therefore, the devices need to be first implanted into the subcutaneous position before one or more pellets can be loaded by hand. This increases the amount of time the incision is open to the air and decreases the sterility of the procedure. It also increases the amount of time the user is holding the device inside the incision with one hand while loading with the other hand, increasing the probability of user error by causing misalignment, contamination, and/or additional trauma to the patient.

Particular advantages of this pellet insertion device include, but are not limited to, development of multiple safety features. For example, an atraumatic cannula that minimizes the chance of internal organ injury and vascular damage yet also prevents the premature egress of the pellet/implant from the distal tip by utilizing the blunt design sectional leaves in a neutral position.

This blunt, atraumatic cannula tip with sectional leaves is vastly superior to the “needle-like” tips of prior art. The rounded, blunt tip minimizes vascular injury by pushing vascular structures to the side during cannula advancement instead of cutting through them. During the boring phase of the procedure with a blunt design, the tissue pressure will place a lateral force on the distal tip of the cannula which stabilizes the tip and prevents a tissue drag. This design also minimizes organ injury by deflecting such structures away from the cannula tip.

The sectional leaves of this blunt, atraumatic tip are a major advantage over prior art and currently available devices. Prior art cannula distal tips have apertures that are completely open, allowing egress of the loaded pellets at any time during the procedure. This allows user error to cause misalignment during the insertion procedure and lead to complications. For example, many prior art devices require the user to insert each pellet individually with one hand, while using the other hand to steady the device while the cannula is inserted into the patient. The sectional leaves on the cannula distal tip of this device greatly reduce the chance of user error and misalignment by preventing the pellets from being inserted into the patient's subcutaneous tissue until the user is completely ready. At the exact moment the user knows the pellets are in position, the spring-loaded cannula is retracted using a button on the top cover of the device. The obturator remains in place with one or more pellets loaded in front of it. The sectional leaves of the cannula distal tip open as the cannula is withdrawn, allowing the pellets to be placed exactly where the user intends.

Another safety feature is the incorporation of a retractable lancet with a depth gauge attached to a lancet housing onto the top cover of the pellet insertion device. The retractable lancet uses a depth gauge to control the initial incision, unlike prior art incisions with scalpels.

In a separate embodiment a lancet is attached to a lancet housing. A lancet cover is then removably attached over the lancet with a safety locking feature, and uses a depth gauge to act as a soft tissue guide during cannula insertion. The lancet cover prevents the cannula from being inserted more than approximately two centimeters perpendicularly. As the user continues to insert the cannula, the lancet cover forces the user to rotate the angle of insertion from perpendicular to horizontal. The combination of these two safety parameters makes this a unique device with a greater safety profile than comparable devices currently available.

Yet another improved safety feature is the integrated cannula and cannula-holder with a loading well that allows for placement of pellets/implants directly into the cannula hollow passageway in a sterile fashion. This is achieved by creating an integrated locking mechanism which allows for pellets packaged adjacently in a flat strip to be secured in a unidirectional fashion and slid over the loading well aperture of the cannula-holder. This innovative design allows for several pellets/implants to be placed sterilely into the loading well and stacked in a vertical fashion to allow advancement directly into the cannula hollow passageway by a stylet or obturator. Once the cannula and cannula-holder are advanced forward to compress the spring mechanism and locked, the loading well is automatically closed by sliding under the top cover of the device, effectively preventing external exposure and possibility of contamination.

The size of the loading well and cannula diameter can vary in different lengths and widths to accommodate the various dimensions of pellets and implants. For this reason, pellets/implants can be placed directly into the loading well under sterile conditions by hand without using pre-packaged pellets/implant in a flat blister packaging strip. This improves universal utilization for a wide variety of pellet/implant manufacturers.

Therefore, it would be beneficial to provide an improved device and blister packaging strip, thereby enhancing the loading and subcutaneous insertion methods to prevent pellet/implant crushing, misalignment, contamination, or extrusion, while simultaneously causing little or no trauma to the subcutaneous tissue and organs.

### SUMMARY

The subject matter of examples of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

A blunt, atraumatic insertion device for subcutaneous delivery of medicated pellets and/or implantable devices is described. The insertion device includes a cannula with a blunt, atraumatic distal tip curving inward in the direction of the longitudinal axis of said cannula. The distal tip has at least three straight-cut sections along the longitudinal axis, resulting in a corresponding number of distal inward-curving tip sections. These sections are bendable, allowing an obturator, at least one pellet, and/or at least one implantable device to be pushed through the hollow internal passageway of the cannula body and egress from the cannula distal tip aperture. Once the objects forcing open the cannula distal tip sections are removed from the aperture, the bendable sections of the cannula distal tip will resume the original blunt, atraumatic shape.

In another embodiment, the distal tip of the obturator has grooves that are shaped to correspond to the bendable cannula distal tip sections. The obturator tip grooves align with the cannula tip sections as the obturator is pushed through the inside of the hollow cannula passageway. The obturator can egress from the cannula distal tip and/or expel anything aligned ahead of the obturator distal tip from the cannula distal tip aperture.

In yet another embodiment, the cannula distal tip of the insertion device has an additional feature. At least one right angle is cut into the diameter of the cannula at the conclusion of the straight-cut sections along the longitudinal axis of the blunt, atraumatic distal tip. This results in at least one stress relief section on the cannula diameter that is bendable. The pressure required to bend the cannula tip sections is reduced by this perpendicular cut, or T-cut, when compared to cannula tip sections that do not have a T-cut. This allows an obturator, at least one pellet, and/or at least one implantable device to be pushed through the inside of the cannula and egress from the cannula distal tip aperture. Once the objects forcing open the cannula distal tip sections with the T-cuts are removed from the aperture, the bendable sections of the cannula distal tip will resume the original blunt, atraumatic shape.

In a further embodiment, the distal tip of the obturator has grooves that are shaped to correspond to the bendable cannula distal tip sections with the T-cut. The blunt, atraumatic cannula distal tip with at least three straight-cut sections along the longitudinal axis conclude with at least one right angle cut, or T-cut, into the diameter of said cannula, resulting in at least one stress relief section on said cannula diameter that is bendable. This allows an obturator with grooves in said distal tip that are shaped to correspond to the bendable cannula distal tip sections with T-cuts to be pushed through the hollow inside passageway of said cannula and egress from the cannula distal tip aperture. Moreover, this allows an obturator, at least one pellet, and/or at least one implantable device to be pushed through the inside of the cannula and egress from the cannula distal tip aperture.

In yet a further embodiment, a T-cut is further cut to create a triangular opening in the cannula distal tip. The insertion device includes a cannula with a blunt, atraumatic distal tip curving inward in the direction of the longitudinal axis of said cannula. The distal tip has at least three straight-cut sections along the longitudinal axis, resulting in a corresponding number of distal inward-curving tip sections. At least one perpendicular angle is cut into the diameter of the cannula at the conclusion of the straight-cut sections along the longitudinal axis of the blunt, atraumatic distal tip. This perpendicular cut, or T-cut, is further cut to result in at least one triangular cut opening along the longitudinal axis, having the triangular cut opening base along the cannula diameter and triangular tip pointing towards the cannula distal tip. This results in a corresponding number of distal inward-curving tip sections. These sections are bendable, allowing an obturator, at least one pellet, and/or at least one implantable device to be pushed through the hollow inside passageway of the cannula body and egress from the cannula distal tip aperture. Once the objects forcing open the cannula distal tip sections are removed from the aperture, the bendable sections of the cannula distal tip will resume the original blunt, atraumatic shape.

In still another embodiment, the distal tip of the obturator has grooves that are shaped to correspond to the bendable cannula distal tip sections with the triangular cut opening. The blunt, atraumatic cannula distal tip with at least three triangular cut-out areas along the longitudinal axis, with the base of the triangular cut in the diameter of the cannula body, resulting in at least one stress relief section on said cannula diameter that is bendable. This allows an obturator with grooves in said distal tip that are shaped to correspond to the bendable cannula distal tip sections with triangular cut openings to be pushed through the hollow inside passageway of said cannula and egress from the cannula distal tip aperture. Likewise, this allows an obturator, at least one pellet, and/or at least one implantable device to be pushed through the inside of the cannula and egress from the cannula distal tip aperture.

In one illustrative embodiment, the invention comprises a device for subcutaneously inserting pellet-shaped medicaments and/or implantable devices into human or animal patients. The invention comprises a cannula with a blunt, atraumatic distal tip curving inward in the direction of the longitudinal axis of said cannula wherein said distal tip has at least three straight-cut sections along the longitudinal axis, resulting in distal inward-curving tip sections, and an aperture on the proximal end large enough for an obturator to travel along the longitudinal axis throughout the hollow passageway inside the cannula body; a cannula-holder, attached to proximal end of said cannula, with an aperture perpendicular to said cannula's longitudinal axis that is large enough for the ingress of a single pellet or insertable implant and which aligns with the hollow passageway inside said cannula body, and a grip attached through the top cover of said device which allows the user to move the cannula-holder along the longitudinal axis of the cannula within the outer body; a spring mechanism located between the cannula-holder and the front cover of the device; a catch-release mechanism, allowing the cannula-holder and the top cover of the device to be engaged when the spring mechanism is compressed; an outer body, comprising a housing and a front cover with an aperture for the distal tip of the cannula, a back cover with an aperture for an obturator with a releasable locking mechanism, a top cover with an aperture located between a proximal clip and a distal clip used to releasably slide a flat packaging strip of pellets or insertable implants, and an aperture that allows the cannula-holder grip to traverse longitudinally within the outer body; an obturator, with a releasable locking mechanism that interacts with the back cover, and a grip on the proximal end; and a strip with flat edges containing one or more pellets and/or insertable implants packaged adjacent to each other that can be removably inserted into at least two corresponding adjustable clips located on opposite sides of an aperture in the top cover of said insertion device.

In another illustrative embodiment, the insertion device includes an attached lancet housing in the distal end of the top cover. In a further illustrative embodiment, the distal end of the top cover includes an attached lancet housing that has an adjustable cover removably attached with a safety locking mechanism.

In yet another illustrative embodiment, the insertion device includes an obturator that has grooves in the distal tip that are shaped to correspond to the cannula distal tip sections, allowing said obturator distal tip to be pushed through the inside of said cannula and egress from said cannula distal tip.

In yet a further illustrative embodiment, the insertion device includes an outer body that has at least one finger grip for the user. In addition to the finger grip, the outer body, top cover, and back cover utilize a series of alternating ridges to assist the user's grasp of the device. The cannula-holder grip, lancet housing, and lancet cover also utilize a series of alternating ridges to assist the user's grip of these components.

In a particular embodiment, a packaging strip for storing and dispensing pellets/implants comprises: a pellet tray top, with at least one capsule-shaped blister packaging cavity arranged on a flat strip; a pellet/implant, located in at least one capsule-shaped blister packaging cavity; a lidding layer of material, attached to the underside of the pellet tray top in order to contain the pellet/implant inside the capsule-shaped blister packaging cavity and comprised of a material that can be opened, allowing the pellet/implant to egress; and a raised area, located on the flat strip and aids in the alignment of the capsule-shaped blister packaging cavity above a targeted egress location.

In another particular embodiment, the packaging strip further comprises a pellet tray bottom attached to the lidding layer of material. The pellet tray bottom has at least one cut-out area to correspond with the capsule-shaped blister packaging cavity. This allows the pellet/implant to egress when the lidding layer material is opened.

In yet another particular embodiment, the packaging strip further comprises a pellet tray bottom attached to the lidding layer of material in which the pellet tray bottom has at least one cut-out area with a serrated edge along a portion of the perimeter that corresponds with the capsule-shaped blister packaging cavity. This allows the pellet/implant to egress when the lidding layer is opened along the serrated edge of the pellet tray bottom.

In a further particular embodiment, the packaging strip is marked so that the user knows which side of the packaging strip should be inserted into the distal clip and the proximal clip of the top cover of the device. This leading edge of the packaging strip is designed for two advantages. First, the serrated perimeter edge of the pellet tray bottom cut-out is located on the non-leading edge of the capsule-shaped blister packaging cavity. As the user pushes down on the top of the capsule-shaped blister packaging cavity, the lidding layer will open first along the serrated edge of the pellet tray bottom on the non-leading edge. The packaging strip is then advanced by sliding it through the distal and proximal clips of the top cover. The leading edge of the torn lidding layer material remains intact, and the tear along the non-leading edge of the lidding layer material is smoothed out as the packaging strip is advanced. This prevents any lidding layer material from falling into the loading well of the cannula-holder during the insertion procedure.

The second advantage is revealed in yet another embodiment of the packaging strip, which comprises a flattened area on the capsule-shaped blister packaging cavity. A typical capsule shape is a bisected sphere with an elongated cylinder connecting the bisected sphere ends. The capsule-shaped blister packaging cavity resembles this shape and is connected to the flat strip along the length of the cylinder portion. The flattened area on the capsule-shaped blister packaging cavity results in a cavity shape that destroys the perfect symmetry of a capsule. It resembles the shape of a malleable capsule that has an external force being applied towards the internal axis of the cylinder portion. In a preferred embodiment, the flattened area is located on the leading edge of the capsule-shaped blister packaging cavity. This design will lead a user to apply external pressure along this area in order to expel the capsule inside. This flattened area feature coupled with the serrated edge along the non-leading edge of the pellet tray bottom will consistently force the tear in the lidding layer material at that exact location. This greatly reduces the chances of any lidding layer material from falling into the loading well of the cannula-holder. If the tear in the lidding layer is located along the perimeter of the leading edge of the capsule-shaped blister packaging cavity, there is a greater likelihood of the lidding layer material bunching up as the packaging strip is advanced. The force of advancing the packaging strip could tear the bunched-up lidding layer material, causing it to fall into the cannula. If the user is unaware of this fact, the lidding layer material could be inserted into the patient along with the pellets/implants and create complications for the patient.

In addition, a novel method for loading pellets and/or implantable devices from a sealed, sterile packaging strip into an insertion device is presented. A method for inserting at least one packaged pellet/insertable implant into an insertion device comprises: inserting a packaging strip with flat edges containing one or more pellets/implants packaged adjacent to each other that can be removably inserted into at least two corresponding adjustable clips located on opposite sides of an aperture in the top cover of said insertion device wherein a cannula has a corresponding aperture perpendicular to the longitudinal axis of said cannula with dimensions large enough for the ingress of at least a single pellet/implant; aligning a pellet/implant packaged in said strip directly above said cannula aperture using raised areas on said strip that correspond to corresponding recessed areas in at least one said adjustable clip; and expelling said pellet/implant from said strip by a user. The pellet/implant strip is preferably packaged in a sterile manner. In another embodiment, the aperture in the top cover of said insertion device and said cannula's aperture located perpendicular to said cannula's longitudinal axis are connected on all sides, forming a loading well. In yet another embodiment, the adjustable clips can accommodate various widths of packaged pellet/implant strips by sliding at least one clip away from said top cover aperture so that the pellet/implant packaging aligns directly above said cannula aperture. In a preferred embodiment, the loading well aperture is slid under the top cover of said insertion device when the cannula is pushed forward, thereby closing it from potential unsanitary particles. In a further embodiment, the packaging strip is released from said top cover aperture clips when the distal clip, attached to said cannula-holder grip, is pushed forward to close said loading well. In yet a further embodiment, at least one raised area on said packaging strip has a rectangular shape which corresponds to a recessed rectangular shape on the distal clip, proximal clip, or both clips.

### FIGURES

The presently disclosed subject matter will be more fully understood by reference to the following drawings which are presented for illustrative, not limiting, purposes.

FIG. 1A is a perspective view of an illustrative cannula with a pellet emerging from three opening distal tip sections, in accordance with embodiments of the disclosure.

FIG. 1B is a perspective view of an illustrative cannula with four straight cut distal tip sections that conclude with a perpendicular cut along the diameter of the cannula, in accordance with embodiments of the disclosure.

FIG. 1C is a perspective view of an illustrative cannula with four distal tip sections separated by triangular cut-out areas, in accordance with embodiments of the disclosure.

FIG. 2 is perspective view of an illustrative obturator with three grooves in the distal tip, in accordance with embodiments of the disclosure.

FIG. 3 is a perspective view of an illustrative cannula-holder, in accordance with embodiments of the disclosure.

FIG. 4 is an exploded view of an illustrative insertion device, in accordance with embodiments of the disclosure.

FIG. 5 is a top elevation view of an illustrative insertion device, in accordance with embodiments of the disclosure.

FIG. 6 is a side elevation view of an illustrative insertion device, in accordance with embodiments of the disclosure.

FIG. 7 is a front elevation view of an illustrative capsule-shaped blister packaging strip with a single pellet, in accordance with embodiments of the disclosure.

FIG. 8 is a transverse cross-sectional view of the illustrative capsule-shaped blister packaging strip with a single pellet shown in FIG. 7, in accordance with embodiments of the disclosure.

FIG. 9 is an exploded view of an illustrative capsule-shaped blister packaging strip, in accordance with embodiments of the disclosure.

FIG. 10 is a top elevation view of an illustrative pellet tray bottom cut-out with serrated edges, in accordance with embodiments of the disclosure.

### DESCRIPTION

In the following description, various examples of the present invention will be described. For purposes of explanation, specific configurations and details are set forth to provide a thorough understanding of the embodiments. However, it will also be apparent to one skilled in the art that the invention may be practiced without the specific details. Furthermore, well-known features may be omitted or simplified so as not to obscure the example being described. The phrase “skin piercing element” as used herein is used in a context which covers not only traditional lancets, but also other skin piercing or skin entering elements including, for example, scalpels, trocars, pointed cannulas, capillary needles, drive pins, or a pioneer projectile. The term “pellet” as used herein covers any subcutaneous object that can be inserted with the invention including, for example, compressed medicaments in the shape of a rice grain or radio frequency identification microchips.

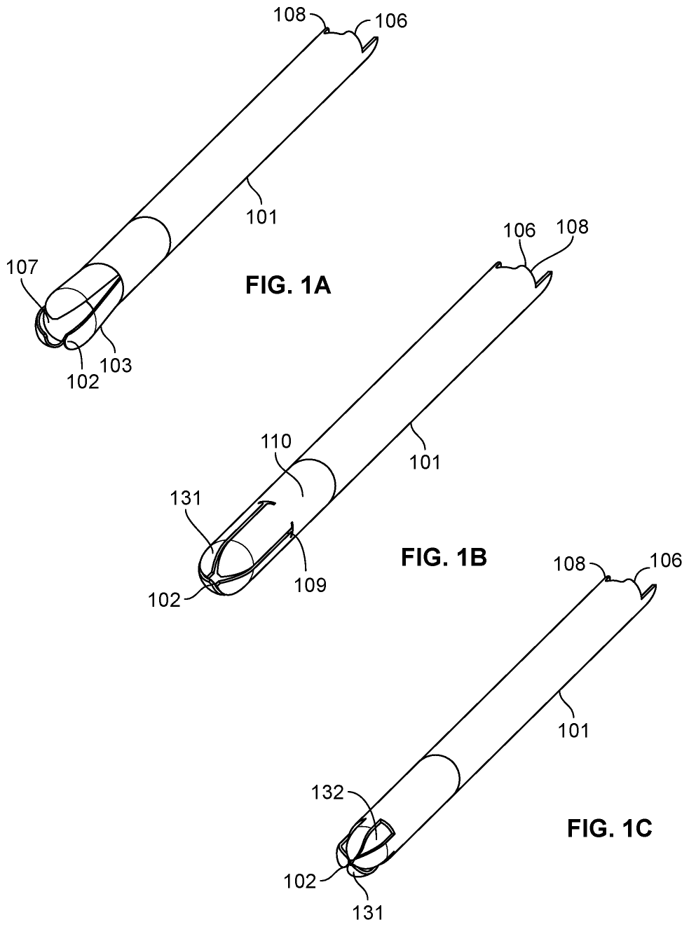

Described herein are insertion devices, component parts, and related methods of function designed to increase the safety and efficacy of inserting sterile medicament pellets and/or implantable devices subcutaneously into a human or animal patient. Referring now to the drawings, in which like reference numerals represent like parts throughout the several views, FIG. 1A, FIG. 1B, and FIG. 1C show perspective views of a cannula body 101 with novel blunt, atraumatic distal tips 102. The combination of a blunt, atraumatic tip that can both bore into tissue and open to eject medicament pellets 107 also eliminates the need for a separate trocar to make the initial bore to the desired subcutaneous location before a pellet can be inserted. The blunt tip of this embodiment reduces the amount of trauma caused by the insertion of the device 100 subcutaneously unlike a sharp instrument, such as a deflected point cannula tip.

The illustrative cannula body 101 of FIG. 1A has at least two apertures, one in the distal tip 102 and one in the proximal end 108. The distal tip 102 of the cannula body 101 has a blunt, atraumatic shape trisected into three equal parts, or tri-leaves 103. In one embodiment the obturator distal tip 104 is first inserted through the proximal aperture 108 of the cannula body into the hollow passageway 106 inside. The tri-leaves 103 open when the distal tip 104 of the obturator 105 applies pressure from the inside of the tri-leaves 103. In another embodiment, the obturator distal tip 104 pushes one or more pellets 107 through the hollow passageway 106 inside the cannula body 101 to open the tri-leaves 103. One or more pellets 107 are thereby ejected coaxially into the linear path created by the cannula distal tip 102 insertion. The ability to simultaneously push multiple pellets 107 through the tri-leaves 103 without causing misalignment or extrusion is a beneficial feature of this invention. In yet another embodiment, the tri-leaves 103 close once the pellet 107 and/or the obturator distal tip 104 is removed from the cannula distal tip 102 opening. In the alternative embodiment, the obturator distal tip 104 pushes through initially to open the tri-leaves 103, which remain open even after the obturator 105 is removed, allowing one or more pellets 107 to be inserted using gravity alone.

Similarly, the illustrative cannula body 101 of FIG. 1B has at least two apertures, one in the distal tip 102 and one in the proximal end 108. The distal tip 102 of the cannula body 101, has a blunt, atraumatic shape quadrisected into four equal parts, or quad-leaves 131. These quad-leaves 131 open when the distal tip 104 of the obturator 105 applies pressure as it pushes through the hollow passageway 106 inside the cannula body 101. In another embodiment, the obturator distal tip 104 pushes one or more pellets 107 through the hollow passageway 106 inside the cannula body 101 to open the quad-leaves 131 and eject one or more pellets 107 coaxially into the linear path created by the cannula insertion. The ability to simultaneously push multiple pellets 107 through the cannula quad-leaves 131 without causing misalignment or extrusion is a beneficial feature of this invention. In yet another embodiment, the quad-leaves 131 close once the pellet 107 and/or the obturator distal tip 104 is removed from the cannula distal tip 102 opening. In the alternative embodiment, the obturator distal tip 104 pushes through initially to open the quad-leaves 131, which remain open once the obturator 105 is removed, allowing one or more pellets 107 to be inserted using gravity alone.

In a further embodiment, the illustrated quadrisected cannula tip has quad-leaves 131 that have been further cut on the perpendicular axis relative to the original longitudinal cut. These perpendicular cuts around the diameter of the cannula, or T-cuts 109, reduce the amount of energy required to force open the quad-leaves 131. The length of the T-cuts 109 determines the size of the “stress-relief” area 110 where the quad-leaves 131 are joined to the cannula. These T-cuts 109 and the resulting stress-relief areas 110 can be modified depending on the fragility of the pellets 107 to be subcutaneously inserted. The purpose of the stress-relief areas 110 is to prevent pellets 107 from being damaged or crushed during the insertion procedure. A damaged pellet 107 will not function as intended, especially ones that are meant to be time-released medicaments that dissolve slowly layer-by-layer.

Likewise, another embodiment of FIG. 1A adds the t-cuts 109 and resulting stress-relief areas 110 to the base of the tri-leaves 103. Additional embodiments are envisioned wherein the blunt, atraumatic distal tip of a cannula has multiple straight cuts along the longitudinal axis of the cannula, resulting in leafy sections that perform similarly to the tri-leaves 103 and/or the quad-leaves 131. If needed, the number of T-cuts 109 and stress-relief areas 110 are determined by the number of cannula distal tip 102 sectional leaves.

The illustrative cannula body 101 of FIG. 1C also has at least two apertures, one in the distal tip 102 and one in the proximal end 108. The distal tip 102 of the cannula body 101, has a blunt, atraumatic shape quadrisected into four equal parts, or quad-leaves 131. Unlike the previously disclosed illustrative cannula distal tips 102, this invention has triangular cut-out sections 132 where the T-cuts 109 would have been located. The base of the triangular cut-out 132 is located on the diameter of the cannula body 101, and the triangular cut-out 132 top is pointing towards the distal tip 102. The distal tip 102 of these quad-leaves 131 open when the distal tip 104 of the obturator 105 applies pressure as it pushes through the hollow passageway 106 inside the cannula body 101. In another embodiment, the obturator distal tip 104 pushes one or more pellets 107 through the hollow passageway 106 inside the cannula body 101 to open the quad-leaves 131 and eject one or more pellets 107 coaxially into the linear path created by the cannula insertion. The ability to push multiple pellets 107 through the cannula quad-leaves 131 eliminates the need to create separate incisions for each pellet 107 to be inserted. In yet another embodiment, the quad-leaves 131 close once the pellet 107 and/or the obturator distal tip 104 is removed from the cannula distal tip 102 opening. In the alternative embodiment, the obturator distal tip 104 pushes through initially to open the quad-leaves 131, which remain open once the obturator 105 is removed, allowing pellets 107 to be inserted using gravity alone.

The second cannula body 101 aperture is located in the proximal end 108. In the preferred embodiment, the cylindrical cannula body 101 is hollow, providing a linear channel through the interior passageway 106 and connecting the distal tip 102 cannula aperture to the proximal cannula aperture 108 along a longitudinal axis. This aperture is large enough for an obturator 105 to fit completely inside the cannula body 101 and traverse longitudinally throughout the length of the hollow passageway 106.

More generally, the preferred cannula distal tip 102 has a tapered shape that may range from 4.0 mm to 6.0 mm in length. The straight cuts to the cannula distal tip 102 to create the tri-leaves 103 or the quad-leaves 131 have a length that may range from 15.0 mm to 18.0 mm. The T-cuts 109 at the base of the tri-leaves 103 or the quad-leaves 131 that run along the cannula body 101 diameter have a length that may range from 0.5 mm to 1.5 mm.

The interior hollow passageway 106 of the cannula body 101 may be sized and shaped to accommodate any suitable pellet 107 or implantable object. Pellets 107 may be any size, shape or type of suitable medical or veterinary formulation. For example, in conventional hormone replacement therapy, hormone pellets 107 for men may range from approximately 3.25 mm to 4.1 mm in diameter, and hormone pellets 107 for women may be approximately 3.0 mm in diameter. Pellets 107 for birth-control applications may be approximately 3.0 mm in diameter. Pellets 107 for agricultural use, such as those for delivering hormones to cattle, range in size from about 2.0 mm to about 5.0 mm. The present invention may be configured with an interior cavity passageway that accommodates any suitable pellet size or shape.

More generally, the preferred cannula body 101 has a length that may range from 13.0 cm up to 17.0 cm. The cannula body 101 length is measured from the distal tip 102 to the proximal cannula aperture 108. More specifically and by way of example and not of limitation, the illustrative tubular cannula body 101 is composed of stainless steel and has an outer diameter 0.50 cm and the hollow passageway 106 inner diameter of 0.45 cm; thus, the wall thickness of the tubular cannula body 101 is 0.05 cm. Additionally, the illustrative tubular cannula body 101 has a length of approximately 15.25 cm.

In another embodiment, the cannula body 101 has at least three apertures. The third aperture is perpendicular to the longitudinal axis of the cannula body 101. It functions as the insertion point of the pellets 107. This perpendicular aperture is large enough for at least one pellet 107 to ingress into the interior hollow passageway 106 of the cannula body 101. In some embodiments, the perpendicular aperture is large enough for multiple pellets 107 to ingress simultaneously into the hollow passageway 106. The perpendicular aperture is preferably attached to the loading well 121 of the cannula-holder 116 near the proximal end of the cannula body 101.

By way of example and not of limitation, the illustrative medication pellet 107 embodiments presented herein may include a 200 mg testosterone pellets 107 that has a 14.00 mm length and a 4.1 mm diameter. An illustrative 100 mg testosterone pellet has a 12.25 mm length and 3.25 mm diameter. The medication pellet may also include estrogen, which is delivered as a tablet having a 3 mm diameter. Thus, the “male” cannula may be sized for 4.1 mm medication pellets 107 for male hormone replacement therapy and the “female” cannula may be sized for 3 mm medication pellets 107. In general, implantable devices for humans are rarely larger than 4.5 mm in diameter.

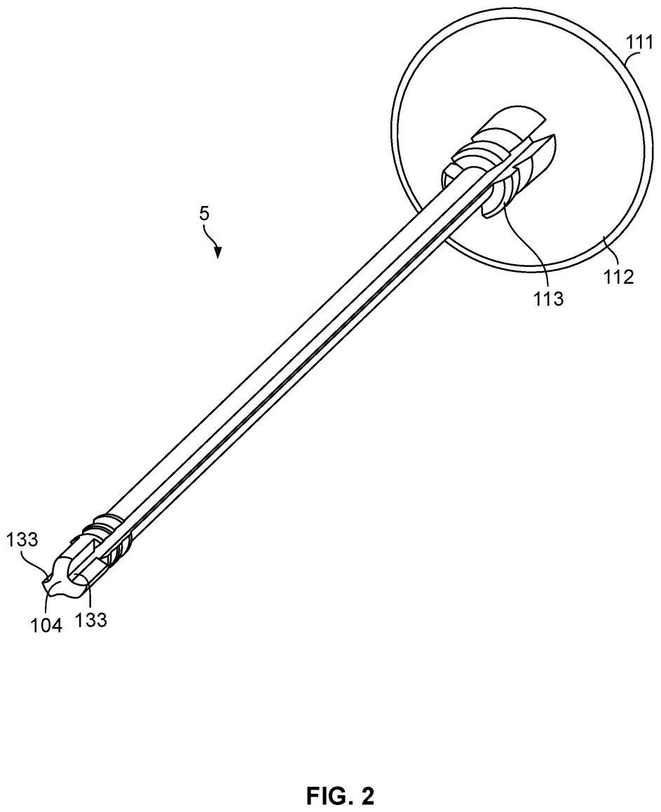

FIG. 2 shows a perspective view of an obturator 105. In a preferred embodiment of the invention, the distal tip 104 of the obturator 105 is designed with grooves 133 that correspond to the shape of the cannula distal tip 102 sectional leaves. In this illustrative embodiment, there are three grooves 133 to correspond with the tri-leaves 103 of FIG. 1A. These grooves 133 allow the obturator distal tip 104 to scrape the tri-leaves 103 clean of any human or animal tissue that might have entered the cannula hollow passageway 106 during insertion into the subcutaneous layer. The number of obturator distal tip 104 grooves 133 depends upon the number of sectional leaves of the cannula distal tip 102. In another embodiment, the cannula distal tip 102 is quadrisected, resulting in quad-leaves 131. The corresponding obturator distal tip 104 in this embodiment has grooves 133 that correspond in shape and size to the cannula distal tip 102 quad-leaves 131.

In the preferred embodiment of the invention, the proximal end 111 of the obturator 105 has a grip 112 to aid in the ergonomic insertion motion. This obturator grip 112 can be a hand grip, finger grip, or combination finger-and-thumb grip. Adjacent to the obturator grip 112 is a releasable-locking mechanism 113 that pairs with the back cover 114 of the outer body 118. The obturator distal tip 104 is inserted into aperture 136 of the back cover 114 and travels along the longitudinal axis of the cannula hollow passageway 106 either before or after the pellets 107 are expelled into the loading well 121 of the cannula-holder 116. The obturator grip 112 is then manually pushed longitudinally by the operator until the releasable-locking mechanism 113 is engaged with the back cover 114 of the device 100. This releasable-locking mechanism 113 is independent of the cannula body 101, which allows the cannula body 101 and attached cannula-holder 116 to be removed from the subcutaneous layer of the human or animal patient while the obturator 105 remains locked in place. The removal of the cannula body 101 prior to the removal of the obturator 105 prevents extrusion of the pellets 107 from the desired subcutaneous target area. Meanwhile, the obturator 105 keeps the boring channel open, which allows the user to assess the level of blood flow egressing from the incision.

In one embodiment, the obturator 105 is longer than the cannula body 101. In another embodiment, the obturator distal tip 104 is not long enough to protrude beyond the cannula distal tip 102 tri-leaves 103 when the obturator coupling mechanism 113 releasably locks with the back cover 114 of the invention when the cannula-holder 116 is releasably attached to the front cover 117 of the outer body 118. There is enough space for at least one pellet 107 to line up in front of the obturator distal tip 104. Once the catch-projection 137 is disengaged from the catch-receiver 157 via the release button 126, the spring mechanism 124 exerts force on the cannula-holder 116 towards the proximal end of the device 100, thereby withdrawing the cannula distal tip 102 tri-leaves 103 while simultaneously expelling at least one pellet 107 and exposing the obturator distal tip 104.

In yet another embodiment, the obturator distal tip 104 protrudes beyond the cannula distal tip 102 tri-leaves 103 when the obturator coupling mechanism 113 releasably locks with the back cover 114 when the catch-projection 137 is engaged with the catch-receiver 157 under the release button 126. The releasable-locking mechanism 113 is transitional between locked and released configurations. The releasable-locking coupling mechanism is selected from a group comprising of a snap-lock coupling, a quick-release spring coupling, snap connectors, mechanical interference fit, or any combination thereof.

In a further embodiment, the releasable-locking mechanism includes a sliding obturator attached to the back cover 114 by a coupling that permits longitudinal reciprocation of the sliding obturator 105 relative to the hollow passageway 106 and prevents rotation of the obturator distal tip 104 relative to the housing passageway 106. This allows the obturator grooves 133 to align correctly with the corresponding sectional leaves of the cannula distal tip 102, such as the tri-leaves 103 or the quad-leaves 131.

By way of example and not of limitation, the preferred obturator 105 has a length that may range from 18.0 cm up to 22.0 cm. The obturator 105 length is measured from the distal tip 104 to the proximal end 111. The designs of the obturator grip 112 and the releasable locking mechanism 113 are both factors that must also be accounted for in the total length calculation.

By way of example and not of limitation, the inner diameter of the cannula hollow passageway 106 is 0.178 inches and the outer diameter of the insertion obturator 105 is 0.167 inches; thus, there is approximately a gap of 0.011 inches between the inner diameter of the cannula hollow passageway 106 and the outer diameter of the insertion obturator 105.

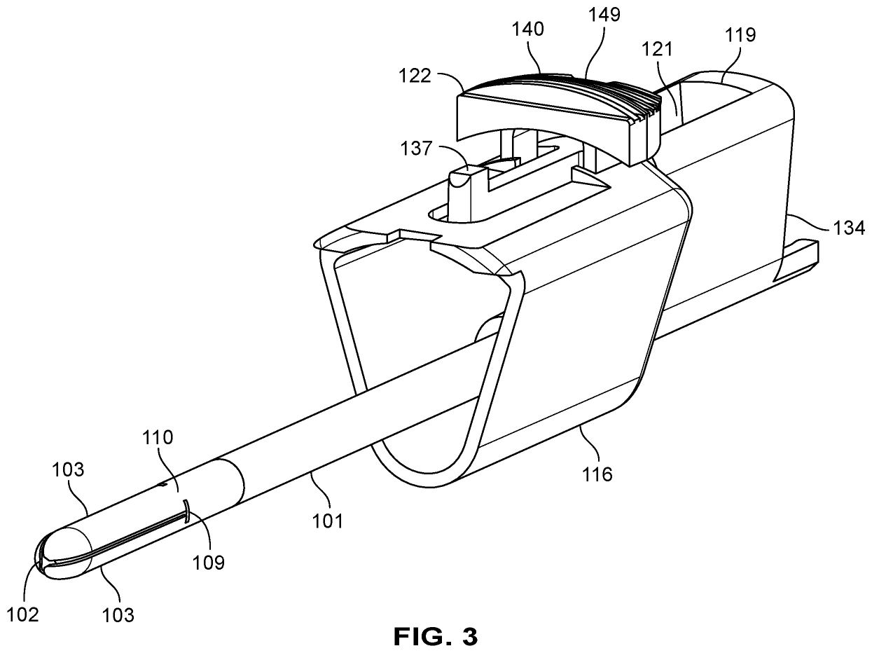

FIG. 3 shows a perspective view of a cannula-holder 116 attached to the proximal portion of the cannula body 101. In the illustrative embodiment, the cannula body 101 has a trisected distal tip 102 with tri-leaves 103, T-cuts 109, and stress-relief areas 110. The proximal cannula aperture 108 is attached to the distal pellet-chamber 115 aperture. The pellet-chamber 115 is an area with the same inner-diameter as the cannula hollow passageway 106 and extends for a length that allows the ingress of at least one pellet 117. The pellet-chamber 115 has a perpendicular aperture to the longitudinal axis of the cannula body 101 that aligns with the cannula-holder aperture 119. The cannula-holder aperture 119 has side walls that frame the outline of the pellet-chamber 115 aperture, creating a loading well 121 in the cannula-holder 116. The pellet-chamber 115 has a proximal aperture 134 that aligns with the back cover 114 aperture 136, allowing the obturator 105 to longitudinally push one or more pellets 107 loaded into the pellet-chamber 115 along the hollow passageway 106 and egress the cannula distal tip 102. In one embodiment, the loading well 121 is designed to stack multiple pellets 107 until the obturator 105 moves the bottom pellet 107 from the pellet-chamber 115 into the cannula hollow passageway 106. The process is repeated until all of the pellets 107 are aligned in front of the distal tip 104 of the obturator 105.

In a preferred embodiment, a grip 122 is attached to the cannula-holder. The grip 122 can be a hand grip, finger grip, or combination finger-and-thumb grip. The cannula-holder 116 is independent of the outer body 118 of the present invention, and is able to move along the longitudinal axis of the cannula 101 within the outer body 118, preferably along a parallel longitudinal sliding track 123 aperture in the top cover 130. The catch-projection 137 of the catch-release mechanism for the spring 124 mechanism is located on the top of the cannula-holder 116, preferably on the distal side of the grip 122.

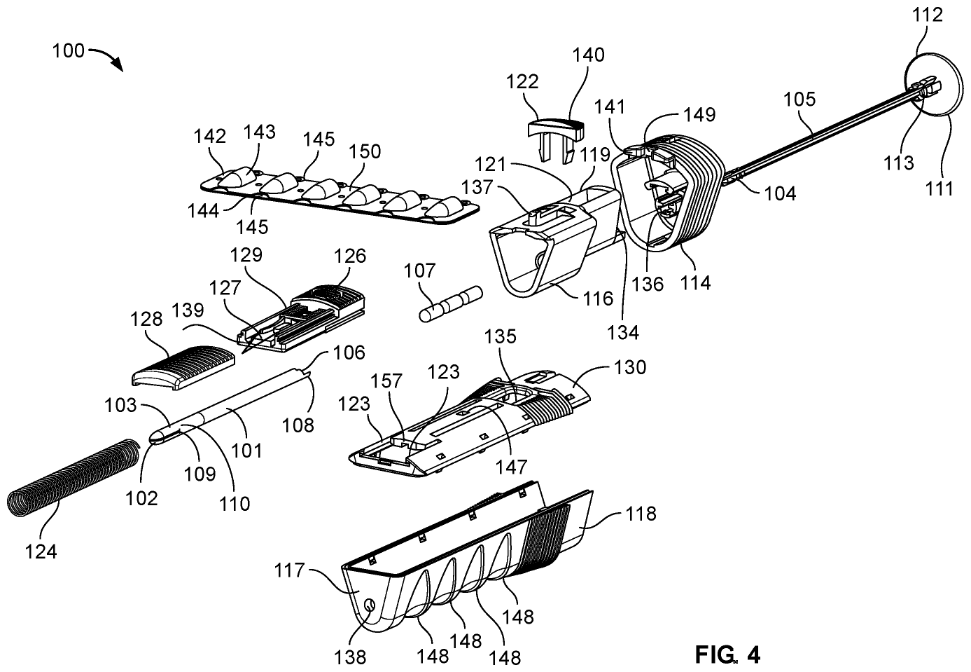

FIG. 4 is an exploded view of a preferred pellet insertion device 100. A compression mechanism, such as a helical spring 124, is located inside the outer body 118 between the cannula-holder 116 and the front cover 117 of the outer body 118. Preferably, a helical spring 124 with a diameter greater than the diameters of both the cannula body 101 diameter and the front cover aperture 138. The helical spring 124 is placed around the cannula body 101 longitudinally between the cannula-holder 116 and the front cover 117 of the outer body 118.

In the illustrated embodiment, a catch-projection 137 of a catch-release mechanism is attached to the cannula-holder 116 and interacts with a catch-receiver 157 located under the release button 126 on the lancet housing 129, which is attached to the distal end of the top cover 130. In a preferred embodiment, the cannula-holder 116 is attached to a grip 122 on the outside of the top cover 130 that allows the cannula-holder 116 to be pushed longitudinally along an aperture, or sliding track 123, in the top cover 130 towards the front cover 117 of the outer body 118, thereby compressing the helical spring 124. The catch-projection 137 engages with the catch-receiver 157 located under the release button 126 in a locked position, securing the cannula-holder 116 in place while the spring 124 is compressed. In another embodiment, the catch-release mechanism has a release button 126 on or near the cannula-holder grip 122. In the illustrative embodiment, the release button 126 of the catch-release mechanism is integrated into the lancet housing 129. The release button 126 is depressed and pushes the catch-projection 137 out of the catch-receiver 157, releasing the energy stored in the compressed spring 124 to eject the cannula-holder 116 and attached cannula body 101 from the insertion site of the human or animal patient. This automatic pull-out of the cannula distal tip 102 prevents possible trauma to the subcutaneous tissue and incision opening because it reduces user error during the removal procedure. Meanwhile, the obturator 105 remains in place due to its releasable-locking mechanism 113 secured to the back cover 114. This forces the cannula distal tip 102 tri-leaves 103 to open as the cannula body 101 retracts over the pellets 107 and the obturator distal tip 104. One or more pellets 107 remain in the desired subcutaneous location created by the cannula distal tip 102 bore hole, and remain held in place by the obturator distal tip 104. The cannula distal tip 102 never retracts fully into the front cover aperture 138. In another embodiment, the opened tri-leaves 103 of the cannula distal tip 102 have a larger diameter than the front cover aperture 138, thereby preventing the cannula distal tip 102 from retracting fully into the outer body 118. The flexible shaft of the obturator 105 can be withdrawn either by releasing the locking mechanism 113 from the back cover 114 and manipulating the obturator grip 112 to withdraw from the incision site, or slowly lifting the pellet insertion device 100 away from the incision site. In another embodiment, the obturator 105 is automatically released from the back cover 114 when the cannula-holder 116 returns to its original position. In effect, the release button 126 unleashes the energy stored in the coiled spring 124 to cause the cannula holder to be pushed proximally and dislodge the obturator releasable locking mechanism 113 from the back cover 114. The ejection of the cannula distal tip 102 from the incision site is followed in short order by the obturator distal tip 104, but not before the pellets 107 are deposited into the targeted subcutaneous location created by the cannula distal tip 102.

In another embodiment, a lancet 127 is located in a lancet housing 129, which is attached to the outer body 118 near the front cover 117, preferably adjacent to the cannula distal tip 102. In the illustrated embodiment, the lancet housing 129 is attached to the distal portion of the top cover 129. The lancet 127 is a skin piercing element. It is used to create the initial puncture of the cutaneous layer of the human or animal patient prior to the insertion of the cannula distal tip 102 and obturator distal tip 104. An adjustable depth gauge 139 is located on the lancet housing 129 and attached to the rear of the lancet 127 to minimize the trauma to the puncture area and the tissue beneath it. The adjustable depth gauge 139 prevents the lancet 127 from piercing the subcutaneous insertion site deeper than the desired location of the pellets 107 being inserted through the cannula distal tip 102. In one embodiment, the depth gauge 139 has a series of markings that correlate to millimeters or centimeters on it. The depth gauge 139 can be coordinated to a specific measurement marking prior to the insertion procedure and be releasably locked so that the lancet 127 does not pierce deeper than the user desires. This reduces the chance of accidental trauma through user error.

In yet another safety feature, a lancet cover 128 is placed immediately onto the lancet 127 after the initial incision is made. This also reduces the chance of accidental trauma through user error. In the illustrated embodiment, the lancet cover 128 uses a sliding track to releasably attach to the lancet housing 129. In a preferred embodiment, the lancet cover 128 has a locking mechanism to both secure it tightly to the lancet housing 129 as well as utilize the depth gauge 139. The lancet cover 128 doubles as a tissue placement guide during the insertion procedure. The lancet cover 128 prevents the spring-loaded cannula distal tip 102 from boring too deeply into the incision because the lancet cover will bump against the skin adjacent to the incision to act as a barrier. The user can no longer push the device 100 vertically down. However, the user can then apply a lateral force towards the bottom of the device 100, opposite the lancet cover 128. This causes the cannula distal tip 102 to penetrate the tissue in a path parallel to the surface of the skin. This is another safety feature in that it prevents the cannula distal tip from boring vertically too deep to cause internal organ injury or vascular damage. The resulting bore hole allows pellets 107 and/or implantable devices to be securely placed and reduces the chances of misalignment or extrusion.

In the illustrative embodiment, the top cover 130 of the outer body 118 has four apertures. The biggest one is the top cover aperture 135. It is approximately the same length and width as the cannula-holder aperture 119, which is large enough for at least one pellet 107 to ingress, and it is located directly above the loading well 121 of the cannula-holder 116. In the assembled device, the distal clip 140 and the proximal clip 141 are located on opposite ends of the top cover aperture 135. The second and third apertures are long slits used as a sliding track 123 for the cannula-holder grip 122, which is attached to the cannula-holder 116 underneath the top cover 135 inside the outer body 118. The cannula-holder grip 122 slides along the tracks 123 to compress the spring 124 and engage the catch-projection 137 on the cannula-holder 116 with the catch-receiver 157 located below the release button 126 on the lancet housing 129. The fourth aperture is small and located in between the sliding tracks 123. This is the catch-projection aperture 147. The tip of the catch-projection 137 can fit inside the catch-projection aperture 147 to stabilize the cannula-holder 116. In another embodiment, the catch-projection aperture 147 is ideally located a specific distance from the proximal clip 141 so that a standard sized blister packaging strip 142 of pellets 107 can be secured using the distal clip 140, which is attached to the cannula-holder 116 and the catch-projection 137.

In the illustrative embodiment, the top cover aperture 135 is part of a sterile pellet loading system. A distal raised arm groove or clip 140 facing the top cover aperture 135 is attached to the cannula-holder grip 122. A proximal raised arm groove or clip 141 facing the top cover aperture 135 is attached to the back cover 114. In another embodiment, the distal clip is not attached to the cannula-holder grip 122. In yet another embodiment, the distal clip 140 and/or the proximal clip 141 is adjustable so that various sized blister packaging strips 142 of pellets 107 with flat edges 144 can be removably secured between the distal clip 140 and the proximal clip 141. Each pellet 107 is encased in a sterile capsule-shaped blister packaging cavity 143, and arranged adjacent to each other on the blister packaging strip 142. Once a capsule-shaped blister packaging cavity 143 is aligned directly above the loading well 121, the user pushes the pellet 107 through the underside of the sterile packaging strip 142, thereby allowing gravity to drop the pellet 107 into the loading well 121 and then the pellet-chamber 115. If more than one pellet 107 is desired, then the blister packaging strip 142 is slid over so that another capsule-shaped blister packaging cavity 143 is aligned above the loading well 121, and the process is repeated. The obturator distal tip 104 is then used to push each pellet 107 from the pellet-chamber 115 into the hollow passageway 106 of the cannula body 101. If more than one pellet 107 is stacked in the loading well 121, then the obturator 105 is withdrawn until the next pellet 107 has descended into the pellet-chamber 115, and the process is repeated until all of the pellets 107 are located in the hollow passageway 106 of the cannula body 101.

In one embodiment, the sterile packaging comprises a strip 142 of pellets 107 individually arranged in capsule-shaped blister packaging cavities 143 designed for asymmetric pressure distribution when a user pushes down on the capsule-shaped blister packaging cavity 143. The lidding layer 152 attached to the underside of the capsule-shaped blister packaging cavity 143 is designed for a limited tear directly below the pellet 107 when the blister is pressed upon by the user. In another embodiment, the lidding layer 152 is designed with a weakened area located at the point or axis of maximum pressure allowing expulsion of the pellet 107 into the loading well 121 of the pellet-chamber 115 without any lidding layer 152 material debris.

In another embodiment, the distal clip 140 is attached to the cannula-holder grip 122. Therefore, the act of pushing the grip 122 forward will compress the spring 124 and allow various sized strips 142 to be fitted between the distal clip 140 and the proximal clip 141. The compressed spring will push the distal clip 140 towards the proximal clip 141, securing the packaging strip 142 in place. Once all of the desired pellets 107 are loaded in the cannula hollow passageway 106, the grip 122 is pushed forward to compress the spring 124 and engage the catch-projection 137 with the catch-receiver 157, locking the cannula-holder 116 in the loaded position. This action causes the blister packaging strip 142 of pellets 107 to fall out of its secured location between the distal clip 140 and the proximal clip 141. Simultaneously, the loading well 121 of the cannula-holder 116 slides under the top cover 130 and prevents anything else from entering into the cannula hollow passageway 106.

In yet another embodiment, the packaging strip 142 has intermittent raised areas 145 along the flat edge 144. These raised areas 145 have complementary recessed areas 149 located in the distal clip 140, proximal clip 141, or both. The alignment of the raised areas 145 with the complementary recessed areas 149 position the capsule-shaped blister packaging cavity 143 directly over the top cover aperture 135 and the loading well 121. The design of the capsule-shaped blister packaging cavity 143 may include a flattened area 150 that allows the user to push down and expel the pellet 107.

In the illustrative embodiment, the obturator 105 is aligned to enter the back cover aperture 136. The obturator distal tip 104 has grooves 133 that align with the back cover aperture 136 and the tri-leaves 103 of the cannula distal tip 102. To insert the obturator 105, the user would only manipulate the proximal end 111 where the obturator grip 112 is located. The obturator releasable locking mechanism 113 interacts with back cover aperture 136 to secure the obturator 105 during the insertion procedure.

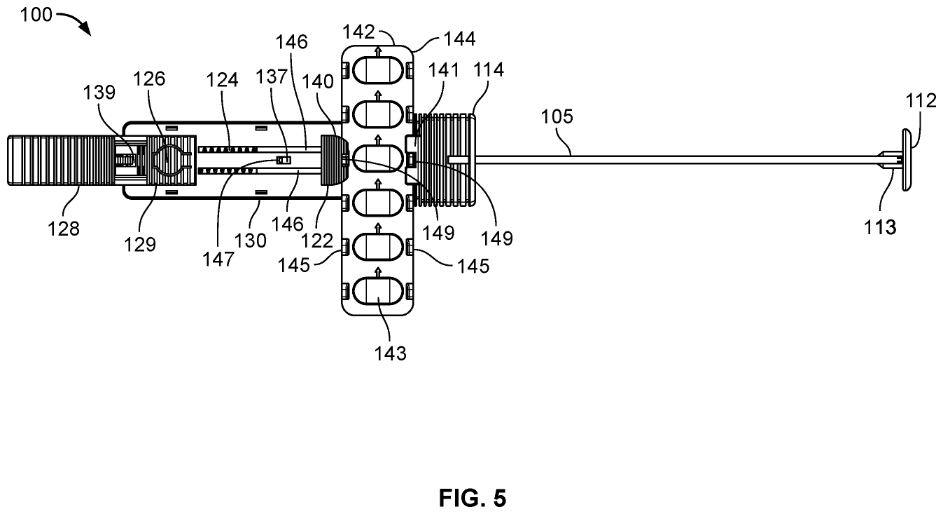

FIG. 5 is a top elevation view of an insertion device 100 with a blister packaging strip 142 of pellets 107 releasably locked into place by the distal clip 140 and the proximal clip 141. The flat edge 144 of the packaging strip 142 has periodically spaced rectangular raised edges 145 that fit into complementary rectangular recessed spaces 149 in the distal clip 140 and proximal clip 141. This feature aligns the capsule-shaped blister packaging cavity 143 directly over the top cover aperture 135, and the loading well 121 in the cannula-holder 116. Various other methods are envisioned to help align the capsule-shaped blister packaging cavity 143 and secure the packaging strip 142 of pellets 107.

In this embodiment, the distal clip 140 is part of the cannula-holder grip 122, which is attached to the cannula-holder 116 through the two slit track 123 apertures in the top cover 130. The cannula-holder grip 122 is pushed toward the distal tip of the device 100 compressing the spring 124 located under the top cover 130. This feature allows the distal clip 140 to adjust for various sized blister packaging strips 142 of pellets 107.

In yet another embodiment, the top cover 130 has an aperture 147 that allows the catch-projection 137, which is attached to the cannula-holder 116, to help secure the cannula-holder grip 122 in place while it fastens the blister packaging strip 142 of pellets 107. When the cannula-holder grip 122 is pushed all the way forward, the catch-projection 137 will engage with the catch-receiver 157 and store the energy in the compressed spring 124, located under the top cover 130. The release button 126 is part of the lancet housing 129 and located directly above the catch-receiver 157. The lancet cover 128 is removably attached to the lancet housing 129, and currently covering the lancet 127 underneath. The depth gauge 139 on the lancet housing 129 is located between the release button 126 and the lancet cover 128.

In the illustrative embodiment, the obturator 105 is only inserted into the back cover aperture 136. The obturator distal tip 104 is not inserted deep enough to interfere with the pellet 107 loading procedure. The pellet 107 in the capsule-shaped blister packaging cavity 143 is ejected from the blister packaging strip 142 and dropped into the loading well 121 of the cannula-holder 116. Once the desired number of pellets 107 are loaded into the loading well 121, the user grasps the obturator grip 112 and fully inserts the obturator 105 into the device, engaging the obturator releasable coupling mechanism 113 with the back cover 114.

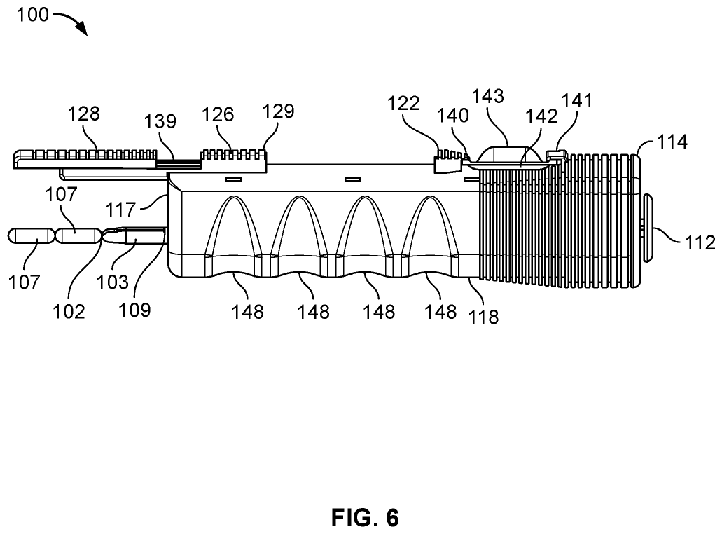

FIG. 6 is a side elevation view of an insertion device 100. An empty capsule-shaped blister packaging cavity 143 on the packaging strip 142 is releasably locked into place by the distal clip 140 and the proximal clip 141. In this embodiment, the insertion device has just released two pellets 107 from the cannula distal tip 102. The cannula-holder grip 122 has returned to its original position adjacent to a separate distal clip 140. This allows the distal clip 140 and proximal clip 141 to securely hold the empty packaging strip 142 in place during the insertion procedure. Prior to the release of the pellets 107, the cannula-holder grip 122 was slid across the length of the top cover 130 and the catch-projection 137 engaged with catch-receiver 157, located under the release button 126 in the lancet housing 129. The lancet housing 129 also contains the depth gauge 139, which is used to precisely adjust the position of the lancet cover 128 in relation to the cannula distal tip 104 and act as a tissue guide during the insertion procedure. The obturator 105 is still inserted into the back cover aperture 136 and the releasable locking mechanism 113 with the back cover 114 is engaged, revealing only the obturator grip 112. In this illustrative embodiment, the obturator distal tip 104 is positioned directly inside the tri-leaves 103 of the cannula distal tip 102. The T-cuts 109 of the tri-leaves 103 have bent back to their original position, and they do not retract into the front cover 117 aperture 138 of the outer body 118. In this embodiment, the outer body 148 has four finger grips 148 to assist the user's grasp during the insertion procedure.

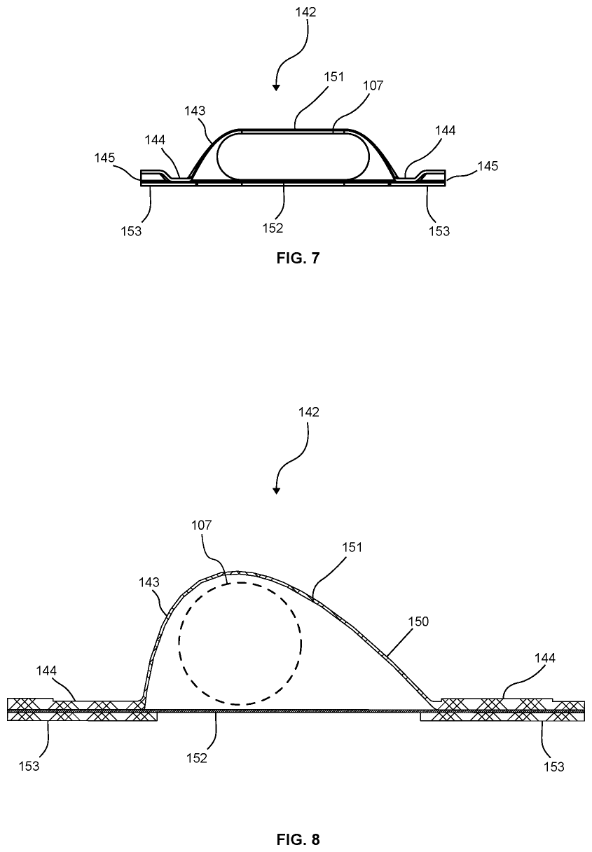

FIG. 7 a front elevation view of an illustrative capsule-shaped blister packaging strip 142. FIG. 8 is a transverse cross-sectional view of the illustrative packaging strip 142 in FIG. 7. In both illustrative embodiments, the pellet tray top 151 comprises a capsule-shaped blister packaging cavity 143 surrounded by a flat strip 144 with rectangular raised areas 145. The flat edges 144 of the packaging strip 142 slide under the distal clip 140 and proximal clip 141. In these illustrative embodiments, the raised areas 145 have a rectangular shape that align with complementary rectangular recessed areas 149 that align the capsule-shaped blister packaging cavity 143 over the loading well 121 of the cannula-holder 116. The pellet 107 is sealed into the capsule-shaped blister packaging cavity 143 with a lidding layer 152 that is attached to the underside of the pellet tray top 151.

In one embodiment, the capsule-shaped blister packaging cavity 143 resembles a geometrical capsule that has been bisected along its length and connected to a flat strip 144. In the illustrated embodiment of FIG. 8, the capsule-shaped blister packaging cavity 143 has a flattened area 150. The flattened area 150 causes the cavity shape to distort the perfectly symmetrical geometrical capsule shape. It now resembles the shape of a malleable capsule that has an external force being applied towards the internal axis of the cylinder portion. In a preferred embodiment, the flattened area 150 is located on the leading edge of the capsule-shaped blister packaging cavity 143. This design will lead a user to apply external pressure along this flattened area 150 in order to expel the capsule 107.

In another embodiment, the blister packaging strip 142 further comprises a pellet tray bottom 153 attached to the underside of the lidding layer material 152. The pellet tray bottom 153 has at least one cut-out area 154 to correspond with the capsule-shaped blister packaging cavity 143. This allows the pellet 107 to egress when the lidding layer 152 is opened.

In yet another embodiment, the blister packaging strip 142 further comprises a pellet tray bottom 153 attached to the lidding layer 152 in which the pellet tray bottom 153 has at least one cut-out area 154 with a serrated edge 155 along a portion of the perimeter that corresponds with the capsule-shaped blister packaging cavity 143. This allows the pellet 107 to egress when the lidding layer 152 is opened along the serrated edge 155 of the pellet tray bottom 153.

In a preferred embodiment, the flattened area 150 feature coupled with the serrated edge 155 along the non-leading edge of the pellet tray bottom 153 will consistently force the tear in the lidding layer 152 material at that exact location. This greatly reduces the chances of any lidding layer 152 material from falling into the loading well 121 of the cannula-holder 116. If the tear in the lidding layer 152 is located along the perimeter of the leading edge of the capsule-shaped blister packaging cavity 143, there is a greater likelihood of the lidding layer 152 material bunching up as the packaging strip 142 is advanced. The force required to advance the packaging strip 142 could tear the bunched-up lidding layer 152 material, causing it to fall into the loading well 121 of the cannula-holder 116. If the user is unaware of this fact, the lidding layer 152 material could be inserted into the patient along with the pellets 107 and create complications for the patient or animal.

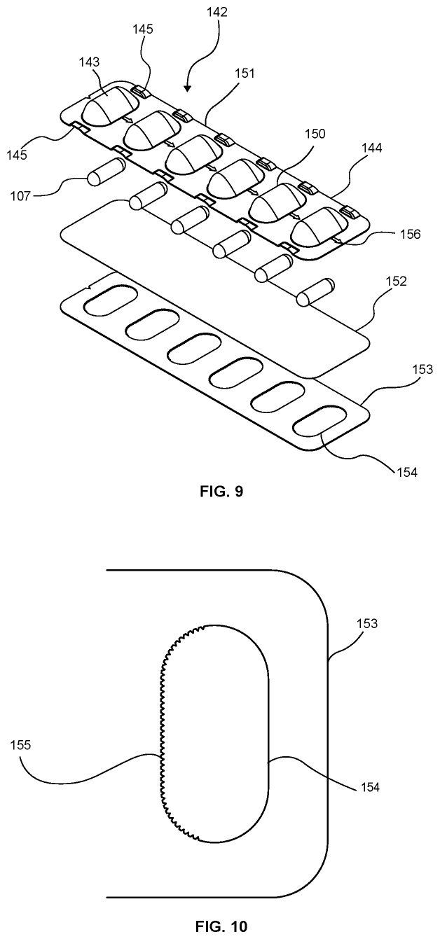

FIG. 9 is an exploded view of the blister packaging strip 142. In this illustrative embodiment, the pellet tray top 151 comprises six capsule-shaped blister packaging cavities 143 surrounded by a flat strip 144 with twelve intermittent rectangular raised areas 145. Arrow marks 156 are used to indicate the leading edge of the flat strip 144 in order to slide it under the distal clip 140 and proximal clip 141 on the top cover 130 of the device 101. The raised areas 145 have a rectangular shape that align with complementary rectangular recessed areas 149 in the distal clip 140 and proximal clip 141 in order to align the capsule-shaped blister packaging cavity 143 over the loading well 121 of the cannula-holder 116. The pellets 107 are sealed into the capsule-shaped blister packaging cavities 143 with a lidding layer 152 that is attached to the underside of the pellet tray top 151. The pellet tray bottom 153 has six capsule-shaped cut-outs 154 that correspond with the six capsule-shaped blister packaging cavities 143. Pressure applied by the user's finger or thumb on the flattened area 150 of a capsule-shaped blister packaging cavity 143 will force a pellet to tear the lidding layer 152 material and expel the pellet 107 through the pellet tray bottom 153 cut-out 154.

FIG. 10 is a top elevation view of an illustrative pellet tray bottom 153 cut-out 154 with serrated edges 155 along a portion of the non-leading edge. The serrated edge 155 resembles sharpened toothlike projections. Each sharpened toothlike projection can create its own small tear in the lidding layer 152 material, reducing the force necessary to create an opening. The purpose of this design is to force the lidding layer 152 material to first tear along the serrated edge 155 instead of elsewhere. If the user requires multiple pellets 107 in the same incision site of a patient, then the user merely adjusts the distal clip 140 and slides the blister packaging strip 142 over to the next available pellet 107 in a capsule-shaped blister packaging cavity 143. Once the rectangular raised areas 145 align with the complementary rectangular recessed areas 149 in the distal clip 140 and proximal clip 141, the spring 124 exerting force in the proximal direction of the device will force the cannula-holder 116 and the attached distal clip 140 to secure the blister packaging strip 142 in place. This method increases the sterility of the insertion procedure because the loading well 121 and cannula hollow passageway 106 remain closed from any potential pathogen or contamination that might enter the device 101 and consequently be introduced inside the patient's body. The only concern with this method is the possibility of some portion of the lidding layer 152 material becoming torn completely off and falling into the loading well 121. This possible concern is greatly diminished when the lidding layer 152 material is only torn along the serration edge 155. The sliding movement of the blister packaging strip 142 forces the torn lidding layer 152 material to smooth out and align with its original flat shape without any additional tearing. In addition, the clear plastic of the capsule-shaped blister packaging cavity 143 will allow the user to ensure that no portion of the lidding layer 152 material is missing.

In the preferred embodiment, both the pellet tray top 151 and pellet tray bottom 153 are composed of a clear polyvinyl chloride (“PVC”), or something suitably similar. The PVC ranges in thickness from approximately 0.25 mm to 0.3 mm. Both the blister packaging strip 142 and pellet tray bottom have a width of approximately 29.0 mm and a length of approximately 110.0 mm. Both accommodate six capsule-shaped blister packaging cavities 143 and complementary capsule-shaped cut-outs 154, each of which is approximately 9.5 mm in width and spaced approximately 18.0 apart as measured from the leading edges. Each capsule-shaped blister packaging cavity 143 has a depth of approximately 5.0 mm as measured from the bottom of the flat edge 144. The serrated edge 155 teeth of the pellet tray bottom cut-out are approximately 0.3 mm in length. The rectangular raised area 145 is approximately 1.1 mm in height as measured from the bottom of the flat edge 144. The leading edges of each rectangular raised area 145 are spaced apart approximately 18.0 mm from each other. The lidding layer 152 is composed of aluminum foil and attached to the pellet tray top 151 and pellet tray bottom 153 with a lacquer or some other suitable substance.

By way of example and not of limitation, each of the components of this invention may be formed from metallic compounds, metal alloys, plastic materials, polymers or other such materials. The material properties may depend on the atraumatic cannula being disposable or reusable. For example, a reusable pellet insertion device 100 may be composed entirely of a stainless-steel material that can be disinfected in an autoclave. The preferred device is intended to be disposed of after a single-use. The cannula body 101 and the lancet 127 are composed of stainless steel. The spring 124 mechanism is composed of spring steel. The remaining pieces are composed of polymer materials. However, it is possible for the preferred invention to be used for more than one insertion of pellets inside the original incision using a method of cannula boring at least fifteen to thirty degrees away from the original deposition of pellets.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “attached” is to be construed as partly or wholly contained within, connected to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Various embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A cannula with a blunt, atraumatic distal tip curving inward in the direction of the longitudinal axis of said cannula, wherein said distal tip has at least three straight-cut sections along the longitudinal axis, resulting in a corresponding number of distal inward-curving tip sections.

2. The cannula of claim 1 wherein at least three straight-cut sections are bendable, allowing an obturator, at least one pellet, and/or at least one implantable device to be pushed through the inside of said cannula and egress from said cannula distal tip.

3. The cannula of claim 1 wherein at least three straight-cut sections are bendable, allowing an obturator with grooves in said distal tip that are shaped to correspond to said bendable cannula distal tip sections to be pushed through the inside of said cannula and egress from said cannula distal tip.

4. The cannula of claim 1 wherein at least one section of said cannula distal tip is bendable when an obturator, at least one pellet, and/or at least one implantable device is pushed through said distal tip sections, and at least one section returns to its original shape when said obturator, pellet, and/or device is no longer prying open said cannula distal tip sections.

5. The cannula of claim 1 wherein at least three straight-cut sections along the longitudinal axis conclude with at least one right angle cut into the diameter of said cannula, resulting in at least one stress relief section on said cannula diameter that is bendable.

6. The cannula of claim 1 wherein at least three straight-cut sections along the longitudinal axis conclude with at least one right angle cut into the diameter of said cannula, resulting in at least one stress relief section on said cannula diameter that is bendable, allowing an obturator, at least one pellet, and/or at least one implantable device to be pushed through the inside of said cannula and egress from said cannula distal tip.

7. The cannula of claim 1 wherein at least three straight-cut sections along the longitudinal axis conclude with at least one right angle cut into the diameter of said cannula, resulting in at least one stress relief section on said cannula diameter that is bendable, allowing an obturator with grooves in said distal tip that are shaped to correspond to said bendable cannula distal tip sections to be pushed through the inside of said cannula and egress from said cannula distal tip.

8. The cannula of claim 1 wherein at least three straight-cut sections along the longitudinal axis conclude with at least one right angle cut into the diameter of said cannula, resulting in at least one stress relief section on said cannula diameter that is bendable, allowing an obturator, at least one pellet, and/or at least one implantable device to be pushed through the inside of said cannula and egress from said cannula distal tip, and said section returns to its original shape when said obturator, pellet, and/or device is no longer prying open said cannula distal tip sections.

9. The cannula of claim 1 wherein the at least three straight-cut sections along the longitudinal axis are further cut to result in at least one triangular cut along the longitudinal axis, having the triangular cut base along the cannula diameter and triangular tip pointing towards the cannula distal tip, resulting in a corresponding number of distal inward-curving tip sections.

10. An insertion device comprising:
a cannula with a blunt, atraumatic distal tip curving inward in the direction of the longitudinal axis of said cannula wherein said distal tip has at least three straight-cut sections along the longitudinal axis, resulting in distal inward-curving tip sections, and an aperture on the proximal end large enough for an obturator to travel along the longitudinal axis throughout the hollow passageway inside the cannula body;
a cannula-holder, attached to proximal end of said cannula, with an aperture perpendicular to the cannula's longitudinal axis that is large enough for the ingress of a single pellet or insertable implant and which aligns with the hollow passageway inside said cannula body, and a grip attached through the top cover of said device which allows the user to move the cannula-holder along the longitudinal axis of the cannula within the outer body;
a spring mechanism located between the cannula-holder and the front cover of the device;
a catch-release mechanism, allowing the cannula-holder and the top cover of the device to be engaged when the spring mechanism is compressed;
an outer body, comprising a housing and a front cover with an aperture large enough for the distal tip of the cannula to egress;
a back cover with an aperture large enough for an obturator with a releasable locking mechanism that engages with the back cover to ingress and egress;
a top cover with an aperture located between a proximal clip and a distal clip that are used to releasably slide a flat-edged packaging strip of pellets or insertable implants, as well as an aperture that allows the cannula-holder grip to traverse longitudinally within the outer body;
an obturator, with a releasable locking mechanism that interacts with the back cover, and a grip on the proximal end of said obturator; and
a packaging strip with flat edges containing one or more pellets and/or insertable implants adjacent to each other that can be removably inserted into at least two corresponding adjustable clips located on opposite sides of an aperture in the top cover of said insertion device.

11. The insertion device of claim 10, further comprising an attached lancet located on the top cover of the device.

12. The insertion device of claim 10, further comprising an attached lancet located on the top cover of the device, said lancet has an adjustable cover removably attached with a safety locking mechanism.

13. The insertion device of claim 10, wherein the distal tip of the obturator has grooves that are shaped to correspond to said cannula distal tip sections, allowing said obturator distal tip to be pushed through the inside of said cannula and egress from said cannula distal tip aperture.

14. The insertion device of claim 10, further comprising at least one finger grip for the user on the outer body.

15. The insertion device of claim 10, further comprising at least one raised area on the flat edge of the blister packaging strip that integrates with a corresponding gap in at least one top cover aperture clip, allowing the packaged pellet/implant to align over the top cover aperture.

16. A packaging strip for storing and dispensing pellets/implants comprising:
a pellet tray top, with at least one capsule-shaped blister packaging cavity arranged on a flat strip;
a pellet/implant, located in at least one capsule-shaped blister packaging cavity;
a lidding layer of material, attached to the underside of the pellet tray top in order to contain the pellet/implant inside the capsule-shaped blister packaging cavity and comprised of a material that can be opened, allowing the pellet/implant to egress; and
a raised area, located on the flat strip that aids in the alignment of the capsule-shaped blister packaging cavity above a targeted egress location.

17. The packaging strip of claim 16, further comprising a pellet tray bottom attached to the lidding layer of material, said pellet tray bottom having at least one cut-out area to correspond with the capsule-shaped blister packaging cavity, allowing the pellet/implant to egress when said material is opened.

18. The packaging strip of claim 16, further comprising a pellet tray bottom attached to the lidding layer of material, said pellet tray bottom having at least one cut-out area with a serrated edge along a portion of the perimeter that corresponds with the capsule-shaped blister packaging cavity, allowing the pellet/implant to egress when said material is opened along said serrated edge.

19. The packaging strip of claim 16, further comprising a flattened area on the capsule-shaped blister packaging cavity.

20. The packaging strip of claim 16, further comprising:
a pellet tray bottom attached to the lidding layer of material, said pellet tray bottom having at least one cut-out area with a serrated edge along a portion of the perimeter that corresponds with the capsule-shaped blister packaging cavity; and
a flattened area on the capsule-shaped blister packaging cavity, allowing a user to apply pressure to said flattened area and force the pellet/implant to open the lidding layer of material at the serrated edge located along the perimeter of the pellet tray bottom cut-out area and egress.
